(19) United States
(12) Patent Application Publication
(10) Pub. No.: US 2011/0059458 A1
(43) Pub. Date: Mar. 10, 2011

(54) COMPOSITIONS AND METHODS FOR CATALYZING DNA-PROGRAMMED CHEMISTRY

(76) Inventors: Yumei Huang, North Billerica, MA (US); James M. Coull, Westford, MA (US); David J. Livingston, Barrington, RI (US)

(21) Appl. No.: 12/918,708

(22) PCT Filed: Feb. 20, 2009
(86) PCT No.: PCT/US2009/034696

§ 371 Date: Nov. 17, 2010

Related U.S. Application Data
(60) Provisional application No. 61/066,701, filed on Feb. 22, 2008.

(51) Int. Cl. C12Q 1/68; C07H 21/04

(52) U.S. Cl. 435/6; 536/25.32

(57) ABSTRACT

The present invention relates compositions and methods that are useful in catalyzing DNA-Programmed Chemistry (or Nucleic Acid-templated chemistry) for use in therapeutic and diagnostic applications.

### CROSS REFERENCE TO RELATED APPLICATION

This application claims the benefit of and priority to U.S. Provisional Patent Application No. 61/066,701, filed Feb. 22, 2008, the entire disclosure of which is incorporated herein by reference.

### FIELD OF THE INVENTION

The present invention relates generally to compositions and methods useful in preparing compounds and compound libraries, preparing detection probes and diagnostic kits. More particularly, the invention relates to compositions and methods that are useful in catalyzing DNA-Programmed Chemistry for use in therapeutic and diagnostic applications.

### BACKGROUND

Catalysts are frequently used to facilitate organic reactions. Recent discoveries indicate that some of the metal catalysts, such as palladium (0) and copper (I), can be used to promote DNA-programmed chemistry (DPC) also known as Nucleic Acid-templated Chemistry (Kanan, M. W.; Rozenman, M. M.; Sakurai, K.; Snyder, T. M.; Liu, D. R. Nature 2004, 431, 545-549). However, certain metal catalysts, such as copper (I), are known to damage and degrade DNA through radical-mediated process (Burrows, C. J.; Muller, J. G. Chem. Rev. 1998, 98, 1109-1152.). Recently, we discovered that small organic molecules such as diamine can be used to facilitate hemicyanine generation through DPC(PCT International Patent Application No. PCT/US2007/021094, titled “Composition and Methods for Biodetection by Nucleic Acid-Templated Chemistry” by Huang et al.). Preliminary studies of the reaction mechanism indicate that those molecules catalyze the DNA template reaction in a different way from the reaction performed in a conventional organic chemical reaction due to the difference in how the reactants approach each other. In a typical DPC reaction, the reactants are attached to oligonucleotides which hybridize under reaction conditions resulting in co-localization of the reactants in a double-stranded DNA format ready to react, while in a conventional organic chemical reaction the reactants move freely in solvent until they collide. Examples of small organic molecules that catalyze conventional organic chemical reactions in water are 1) aniline-catalyzed hydrozone and oxime formation (Dirksen, A.; Dirksen, S.; Hackeng, T. M, Dawson, P. E. J. Am. Chem. Soc. 2006, 128, 15602-15603; Dirksen, A.; Hackeng, T. M., Dawson, P. E. Angew. Chem. Int. Ed. 2006, 45, 7581-7584); 2) Proline analog direct asymmetric aldol reactions (Mase, N.; Nakai, Y.; Ohara, N.; Yoda, H.; Takabe, K.; Tanaka, F.; Barbas III, C. F. J. Am. Chem. Soc. 2006, 128, 734-735.); 3) Small peptide-catalyzed direct asymmetric aldol reaction (Dziedzic, P.; Zou, W.; Háfren, J.; Córdova, A. Org. Biomol. Chem. 2006, 4, 38-40.); and 4) Zinc-proline catalyzed aldol reactions (Kofoed, J.; Darbre, T.; Reymond, J. Chem. Commun. 2006, 1482-1484.).

### SUMMARY OF THE INVENTION

The present invention is based, in part, upon the discovery that an improved nucleic acid-templated reaction (or DNA-programmed chemical reaction) can be achieved using catalysts that are conjugated to one or more of the oligonucleotides involved in the nucleic acid-templated reaction thereby achieving a catalytic effect upon the reaction.

In one aspect, the invention provides a method for detecting a biological target in a sample. The method comprises providing (a) a first probe component comprising (i) a first binding moiety having binding affinity to the biological target, (ii) a first oligonucleotide sequence associated with the first binding moiety, (iii) a first reactive group associated with the first oligonucleotide sequence, and (iv) an optional catalytic moiety associated with the first probe component; and (b) a second probe component comprising (i) a second binding moiety having binding affinity to the biological target, (ii) a second oligonucleotide sequence associated with the second binding moiety, (iii) a second reactive group associated with the second oligonucleotide sequence, and (iv) an optional catalytic moiety associated with the second probe component, wherein the second oligonucleotide is capable of hybridizing to the first oligonucleotide sequence and the second reactive group is capable of reacting with the first reactive group. At least one catalytic moiety is associated with the first probe component or the second probe component, and the catalytic moiety is capable of catalyzing the reaction between the first reactive group and the second reactive group.

Thereafter, the first and second probe components are combined with a sample to be tested for the presence of the biological target under conditions that permit (i) the first binding moiety and the second binding moiety to bind to the biological target, if present in the sample, and (ii) the second oligonucleotide to hybridize to the first oligonucleotide thereby to bring the first reactive group into reactive proximity with the second reactive group. A reaction between the first and the second reactive groups can be detected thereby determining the presence of the biological target in the sample. The same procedure can be used to quantify the amount of the biological target in the sample.

In certain embodiments, the biological target can be a protein or nucleic acid. When the biological target is a protein, in certain embodiments, each of the first binding moiety and the second binding moiety is an antibody that binds to the biological target. When the biological target is a nucleic acid, in certain embodiments, each of the first binding moiety and the second binding moiety is a nucleic acid that is complementary to a portion of the biological target. It is understood that the first and second binding moieties can bind different sites on the biological target.

In certain embodiments, the catalytic moiety is a diamine of the formula (II):

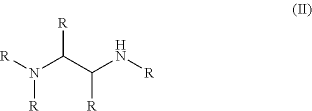

wherein each R is independently selected from hydrogen or C₁-C₆ straight or branched alkyl. Exemplary catalytic moieties can be selected from the group consisting of N¹,N¹-dimethylethane-1,2-diamine, N¹,N¹-dimethylpropane-1,3-diamine, N²,N²-dimethylpropane-1,2-diamine, ethylenediamine, N¹,N¹-diethylethylene-1,2-diamine (“DMEDA”), propane-1,2-diamine, and 1-(2-aminoethyl)-piperidine.

In certain embodiments, each of the first and second probe components is a single molecule where the chemical fragments of each probe component are covalently associated with one another. For example, the first reactive group is covalently associated with the first oligonucleotide, which is covalently associated with the first binding moiety; and the second reactive group is covalently associated with the second oligonucleotide, which is covalently associated with the second binding moiety. Alternatively, one or both of the first and second probe components can comprise a plurality of chemical fragments that are non-covalently associated with one another to produce functional probe components. For example, the probe components can comprise two or more oligonucleotide sequences, for example, a zipcode oligonucleotide sequence and a complementary or substantially complementary anti-zipcode oligonucleotide sequence, which are capable of hybridizing to one another to permit non-covalent association of these chemical species.

In certain embodiments, the catalytic moiety is covalently associated with the first oligonucleotide or the second oligonucleotide. In certain other embodiments, the catalytic moiety is covalently associated with the first reactive group or the second reactive group. Although the precise location of the catalyst may vary, the catalytic moiety should be located within catalytic proximity to the first and second reactive groups so as to catalyze the reaction between the first and second reactive groups.

In another aspect, the invention provides a method of performing a chemical reaction using a nucleic acid template to produce a reaction product. The method comprises providing (i) a template comprising a first reactive group covalently associated to a first oligonucleotide defining a first codon sequence and an optional catalytic moiety covalently associated with the first oligonucleotide or first reactive group, and (ii) a transfer unit comprising a second reactive group covalently associated with a second oligonucleotide defining a first anti-codon sequence complementary to the first codon sequence of the template and an optional catalytic moiety covalently associated with the second oligonucleotide or second reactive group. At least one catalytic moiety is covalently associated with one of the first oligonucleotide, the second oligonucleotide, the first reactive group, or the second reactive group. Thereafter, the template and the transfer unit are combined under conditions so that the first codon sequence and the first anti-codon sequences anneal to one another, which brings the first reactive group into reactive proximity with the second reactive group whereupon the first reactive group reacts with the second reactive group to produce a reaction product. The reaction between the first reactive group and the second reactive group is catalyzed by the catalytic moiety.

In another aspect, the invention provides a method for performing a chemical reaction using a nucleic acid template to produce a reaction product. The method comprises providing (i) a first transfer unit comprising a first oligonucleotide sequence and a first reactive group covalently associated with the first oligonucleotide sequence and an optional catalytic moiety covalently associated with the first oligonucleotide or first reactive group, (ii) a second transfer unit comprising a second, different oligonucleotide sequence and a second reactive group covalently associated with the second oligonucleotide sequence, and an optional catalytic moiety covalently associated with the second oligonucleotide or second reactive group, and (iii) a template comprising a template oligonucleotide sequence and an optional catalytic moiety covalently associated with the template oligonucleotide. The first oligonucleotide sequence and the second oligonucleotide sequence are complementary to two separate regions of the template oligonucleotide. In addition, at least one catalytic moiety is covalently associated with one of the first oligonucleotide, the second oligonucleotide, the first reactive group, the second reactive group, or the template oligonucleotide. Thereafter, the first transfer unit, the second transfer unit and the template are combined under conditions so that the first oligonucleotide and the second oligonucleotide hybridize to their respective complementary regions of the template oligonucleotide to bring first reactive group into reactive proximity with the second reactive group. The first reactive group reacts with the second reactive group to produce a reaction product, and the reaction between the first reactive group and the second reactive group is catalyzed by the catalytic moiety. In other words, rate of reaction between the first reactive group and the second reactive group is faster in the presence of the catalytic moiety than in the absence of the catalytic moiety.

In another aspect, the invention provides a method of performing a chemical reaction using a nucleic acid template to produce a reaction product. The method comprises providing (i) a template comprising a first reactive group covalently associated with a first oligonucleotide defining a first codon sequence, (ii) a transfer unit comprising a second reactive group associated with a second oligonucleotide defining a first anti-codon sequence complementary to the first codon sequence of the template, and (iii) a third unit comprising a catalytic moiety covalently attached to a third oligonucleotide defining a third sequence, wherein the third oligonucleotide is capable of forming a triplex with the first and the second oligonucleotides through Hoogsteen or reversed Hoogsteen hydrogen bonds. Thereafter, the template, the transfer unit, and the third unit are combined under conditions to produce a triplex comprising the first, second and third oligonucleotides whereupon the catalytic moiety, the first reactive group and the second reactive group are brought into proximity with one another so that the first and second reactive groups react with one another to produce the reaction product. The reaction between the first and the second reactive groups is catalyzed by the catalytic moiety.

In another aspect, the invention provides a method for performing a chemical reaction using a nucleic acid template to produce a reaction product. The method comprises providing (i) a first transfer unit comprising a first oligonucleotide sequence and a first reactive group covalently associated to the first oligonucleotide sequence, (ii) a second transfer unit comprising a second oligonucleotide sequence and a second reactive group covalently associated to the second oligonucleotide sequence, (iii) a template comprising a template oligonucleotide sequence, wherein the first oligonucleotide sequence and the second oligonucleotide sequence are complementary to two separate regions of the template oligonucleotide, and (iv) a third unit comprising a catalytic moiety covalently associated with a third oligonucleotide defining a third sequence, wherein the third oligonucleotide is capable of forming a triplex with the first oligonucleotide and the template oligonucleotide through Hoogsteen or reversed Hoogsteen hydrogen bonds. Thereafter, the first transfer unit, the second transfer unit, the template and the third unit are combined under conditions so that (i) the first oligonucleotide and the second oligonucleotide hybridize to their respective complementary regions of the template oligonucleotide to bring the first reactive group into reactive proximity with the second reactive group, and (ii) the first oligonucleotide, the template oligonucleotide and the third oligonucleotide form a triplex to bring the catalytic moiety into proximity with the first and second reactive groups to catalyze the reaction between the first and second reactive groups to produce a reaction product.

In another aspect, the invention provides a method for detecting a biological target in a sample. The method comprises providing: (a) a first probe component comprising (i) a first binding moiety having binding affinity to the biological target, (ii) a first oligonucleotide sequence, and (iii) a first reactive group covalently associated with the first oligonucleotide sequence; (b) a second probe component comprising (i) a second binding moiety having binding affinity to the biological target, (ii) a second oligonucleotide sequence, and (iii) a second reactive group associated with the second oligonucleotide sequence, wherein the second oligonucleotide is capable of hybridizing to the first oligonucleotide sequence and the second reactive group is capable of reacting with the first reactive group when the second reactive group is brought into reactive proximity with the first reactive group; and (c) a third probe component comprising (i) a third binding moiety having binding affinity to the biological target, (ii) a third oligonucleotide sequence, and (iii) a third reactive group reactive with the first and/or the second reactive groups, or a catalytic moiety, wherein the catalytic moiety is capable of enhancing the reaction rate between the first and second reactive groups.

The first, the second and the third probe components are combined with a sample to be tested for the presence of the biological target under conditions so that (i) the first, the second and the third binding moieties bind to the biological target, if present in the sample, and (ii) a triplex forms between the first, the second and the third oligonucleotides to bring the first and second reactive groups into proximity with the third reactive group or the catalytic moiety. A reaction among the reactive groups is detected so as to determine whether the biological target is present in the sample. The same approach can also be used to quantify the amount of the biological target in the sample.

In another aspect, the invention provides an improved method of detecting a biological target in a sample, where the improvement comprises providing a reaction product by one of the foregoing methods, wherein the formation of the reaction product is indicative of the presence of the biological target in the sample.

In another aspect, the invention relates to a diagnostic kit that generates detectable signal by one or more of the above methods. In one specific aspect, the kit comprises in separate containers: (1) a first probe component comprising (i) a first binding moiety having binding affinity to the biological target, (ii) a first oligonucleotide sequence associated with the first binding moiety, and (iii) a first reactive group associated with the first probe component; (2) a second probe component comprising (i) a second binding moiety having binding affinity to the biological target, (ii) a second oligonucleotide sequence associated with the second binding moiety and capable of hybridizing to the first oligonucleotide sequence, and (iii) a second reactive group associated with the second oligonucleotide sequence and reactive with the first reactive group to produce a reaction product when brought into reactive proximity of one another in the presence of a catalytic moiety, wherein the catalytic moiety is associated with at least one of the first probe component or the second probe component; and (3) instructions for using the kit for detecting the biological target.

In certain of the kits, each of the first and second probe components is a single molecule where the chemical fragments of each probe component are covalently associated with one another. Alternatively, one or both of the first and second probe components can comprise a plurality of chemical fragments that are non-covalently associated with one another to produce functional probe components. For example, the probe components can comprise two or more oligonucleotide sequences, for example, a zipcode oligonucleotide sequence and a complementary or substantially complementary anti-zipcode oligonucleotide sequence, which are capable of hybridizing to one another to permit non-covalent association of these chemical species.

Similarly, the catalytic moiety may be covalently associated with any one of the probe components or, alternatively, covalently associated with a separate oligonucleotide sequence that is complementary or substantially complementary with an oligonucleotide sequence present in one of the probe components. In one embodiment, the catalytic moiety is associated with a probe component that comprises one of the reactive groups. In another embodiment, the catalytic moiety is associated with a separate oligonucleotide that is complementary or substantially complementary with a nucleotide sequence present on a probe component that comprises one of the reactive groups.

In certain embodiments, the diagnostic kit further comprises a detectable reagent that specifically associates with the reaction product.

### BRIEF DESCRIPTION OF THE FIGURES

FIG. 1 depicts an embodiment of an “all-in-one” design where a catalyst and the reactants are linked to a bifurcated DNA strand (hereinafter Design A).

FIG. 2 depicts an embodiment of an “all-in-one” design where a catalyst is linked through one of the reactants to a DNA strand (hereinafter Design B).

FIG. 3 depicts an embodiment of an “all-in-one” design where a catalyst and one of the reactants are components of a single chemical fragment that is attached to a DNA strand (hereinafter Design C).

FIG. 4 depicts an embodiment of an “all-in-one” design where a catalyst is linked to a DNA template that templates the reactant DNA strands (hereinafter Design D).

FIG. 5 depicts an embodiment of an “all-in-one” design where a catalyst is linked to a third DNA strand that can form a triplex with a reactant/template strand through Hoogsteen or reversed Hoogsteen hydrogen bonds (hereinafter Design E).

FIG. 6 depicts a proposed mechanism for diamine-catalyzed DPC hemicyanine formation.

FIG. 7 depicts an exemplary DPC reaction to produce a fluorophore in the presence of an EGFR-ErbB2 heterodimer.

FIG. 8 depicts an exemplary synthetic sequence for preparing a bifurcated DNA strand bearing a reactant and catalyst (i.e., a DNA strand of Design A).

FIG. 9 depicts an exemplary synthetic sequence for preparing a sequentially-linked DNA strand bearing a reactant and catalyst (i.e., a DNA strand of Design B).

FIG. 10 depicts an exemplary synthetic sequence for preparing a DNA strand bearing a chemical fragment comprising both a catalyst and a reactant (i.e., a DNA strand of Design C where the reactant that is an aldehyde).

FIG. 11 depicts exemplary reagents for preparing a DNA-small molecule conjugate.

FIG. 12 depicts an exemplary DPC reaction forming a particular hemicyanine compound in the presence of an EGFR-ErbB2 heterodimer.

### DETAILED DESCRIPTION OF THE INVENTION

In its simplest sense, the invention provides methods where nucleic acid-templated reactions are enhanced by catalysts that are directly incorporated in the reaction system.

Although the design of a catalyst (for example, a diamine catalyst) to act in a concerted fashion upon two otherwise unreactive moieties to rapidly catalyze a high yield of product is very attractive, there are some potential disadvantages for this system. First, a millimolar concentration of the catalyst may be needed for efficient hemicyanine formation. This high concentration might not be compatible with or could even be toxic to some biological systems. However, high catalyst concentration may be required for establishing and maintaining a Schiff's base under aqueous conditions throughout the initial reaction sequence in order to localize the catalyst at the reaction site. Second, the reaction rate may not be fast enough for a “point-of-care” device (as the reaction may take 1 to 2 hours for completion). Without wishing to be bound by theory, it is believed that, in the case of diamine catalysts, the slow reaction kinetics is due, in part, to the hydrophilic catalyst's slow diffusion rate to the hydrophobic core of DNA site and cannot be dramatically improved by just optimizing the catalyst's structure. Although increasing the catalyst's concentration increases its diffusion rate, high catalyst concentration destabilizes the indolinium compound and is also toxic for most biological systems. Increasing hydrophobicity of the catalyst decreases its water solubility. Third, catalyst has to be added separately to the reaction mixture and this increases the complexity of the system.

In order to simplify the above system while taking advantages of the catalytic enhancement and minimize the disadvantages, we describe here “all-in-one” designs where catalysts are built into template DNAs and/or reactant DNA strands. Such “all-in-one” designs allow the possibility of synthesis of bioactive compounds and/or reporter compounds in living systems where the introduction of an excess of catalyst are difficult or toxic. Since catalyst, reactants and DNA are all in one catalyzed DPC reaction system, there is no catalyst accessibility issue for the DPC reactants and the reaction rate can increase dramatically. The increased reaction rate benefits reactive reactants such as indolinium compound and minimizes their possible decomposition due to the prolonged reaction time. The elimination of high concentration of catalyst additives also help stabilize the reactant, thus the product yield is increased. These combined effects enable a more sensitive and convenient bioassay for the biological detection than the previous method.

The choice of catalytic moiety for use in the present invention is of course dependent upon the nature of the reactive groups and in particular the type of chemical reaction desired between those reactive groups. In one embodiment, the first reactive group is an aldehyde and the second reactive group is an active hydrogen component. In this embodiment, the catalytic moiety is selected from a secondary amine, a primary amine, a bifunctional amine-acid catalyst or a diamine.

Secondary amines useful as catalytic moieties in this invention include, but are not limited to pyrrolidines such as 1-(2-pyrrolindinylmethyl)-pyrrolidine, piperidines, a nornicotines, prolines, or analogs thereof.

Primary amines useful as catalytic moieties in this invention include, but are not limited to 1-(ethylpyrrolidin-2-yl)methanamine, 2-aminomethylpyrrolidine, valine or a peptide having fewer than 3 amino acid units, or an amino acid of the general formula (I):

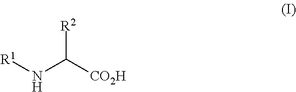

wherein R¹ is hydrogen or C₁-C₆ straight or branched alkyl; and R² is C₁-C₆ straight or branched alkyl.

Bifunctional amine-acid catalysts useful as catalytic moieties in this invention include, but are not limited to pyrrolidine/AcOH.

Diamine catalysts useful as catalytic moieties in this invention include, but are not limited to a compound of the general formula (II):

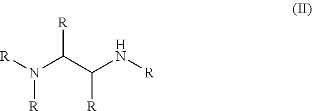

wherein each R is independently selected from hydrogen or C₁-C₆ straight or branched alkyl. In one embodiment, each R is independently selected from hydrogen or C₁-C₆ straight alkyl. Specific examples of diamine catalysts include N¹,N¹-dimethylethane-1,2-diamine, N¹,N¹-dimethylpropane-1,3-diamine, N²,N²-dimethylpropane-1,2-diamine, ethylenediamine, N¹,N¹-diethylethylene-1,2-diamine (“DMEDA”), propane-1,2-diamine, 1-(2-aminoethyl)-piperidine, or an analog thereof, for example. In one specific embodiment, the catalytic moiety is DMEDA.

The reactive groups utilized in this invention react with one another in the presence of the catalytic moiety that is covalently attached to one of the oligonucleotides to produce a product (“reaction product”). In one embodiment the reaction product is a polymethine dye. In one embodiment, the reaction product is a hemicyanine dye. It is understood that the reaction product can be a peptide or a small molecule.

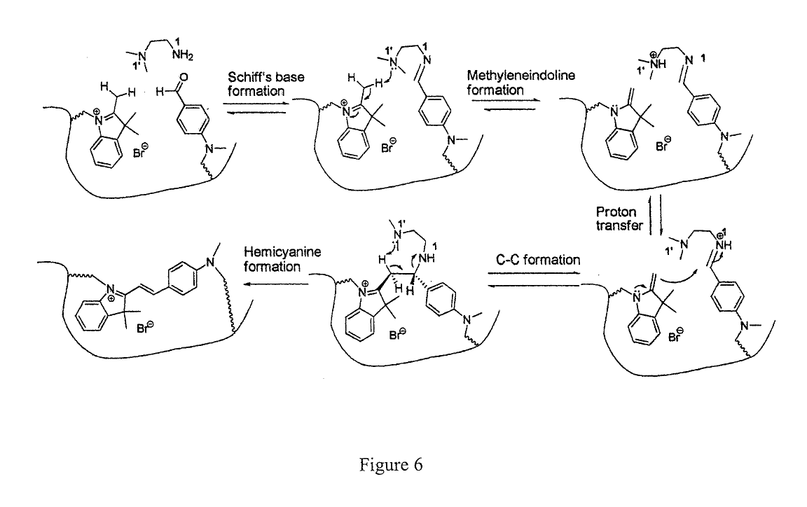

Although not to be bound by a particular mechanistic theory, a possible mechanism for hemicyanine formation is illustrated in FIG. 6. For the purpose of illustration, the catalyst in FIG. 6 is dimethylethane diamine (DMEDA). The proposed mechanism involves the N1-nitrogen atom reacting with the aldehyde to faun a Schiff's base which brings the catalyst into proximity with the indolinium compound. The lone pair electrons on the N1′-nitrogen atom extract a proton from the methyl group of the indolinium compound converting it to the somewhat stabilized methyleneindoline. The proton on the N1′-nitrogen atom is subsequently transferred to the N1-nitrogen atom of the Schiff's base due to its higher pKa. A contributing resonance structure increases the electrophilicity of the imine carbon and makes it susceptible to attack by nucleophiles. As a result, the methyleneindoline then reacts with the protonated imine to form a neutral intermediate which upon intramolecular proton abstraction by N1′-nitrogen atom eliminates the catalyst to form the stable hemicyanine product. Although most of the proposed steps are in equilibrium, the extended conjugation of the hemicyanine product essentially drives the reaction to completion. Addition of NaCNBH₃ to DPC reaction mixtures provided direct evidence for the Schiff's base intermediate, as LC-MS analysis has provided quantitative formation of the reduced Schiff's base (secondary amine formation) for DMEDA.

In some embodiments, the methods described herein are used to detect and/or quantify a biological target, which can include, for example, a protein, peptide, nucleic acid, or carbohydrate. Exemplary proteins include, for example, a receptor, ligand, hormone, enzyme, or immunoglobulin. The biological target can be a multimeric protein, for example, a homodimeric protein, a heterodimeric protein, or a fusion protein.

Exemplary multimeric proteins that can be detected and or quantified, include, for example, ErbB protein family homo- and heterodimers (e.g., ErbB2 (HER2) homodimers, ErbB1 (EGFR) homodimers, EGFR/ErbB2 heterodimers, etc), VEGF receptor homo- and heterodimers, VEGF dimmers, PDGF dimmers, tyrosine kinase receptor complexes, TNF/TNFR complexes, cadherin complexes, catenin complexes, IGFR complexes, insulin receptor complexes, receptor/receptor ligand complexes (e.g., EPO/EPO receptor), NF-kB/IkB complexes, T-cell antigen complexes, integrin protein complexes, FKBP protein complexes, p53 protein complexes, Bcl family protein complexes, Myc/Max complexes, cyclin protein complexes, intracellular protein kinase complexes, caspase protein complexes, autoantibody-antigen complexes, and secreted protein complexes (e.g., amyloid protein complexes. The methods of this invention are particularly useful in the detection and/or quantification of ErbB protein family homodimers or heterodimers.

Exemplary fusion proteins that can be detected and/or quantified, include, for example, Bcr-Abl; NPM-ALK; and certain ALK containing fusion proteins.

Exemplary post-translational modifications that can be detected and/or quantified, include, for example, phosphorylated proteins (e.g., phosphorylated STAT proteins); glycosylated proteins; and farnesylated proteins (e.g., RAS).

Under certain circumstances, the formation of a reaction product requires the use of two probe components that associate with one another to produce a reaction product by DPC that can be detected directly or indirectly. It is understood that individual probe components do not contain a reaction product, but are capable of associating with a complimentary probe component to produce a reaction product by DPC.

It is understood that in the methods of detecting a biological target, the first probe component and the second probe component (and the third probe component in certain embodiments) can each be a single molecule. For example, in the first and second probe components, the binding moiety can be covalently bound to the oligonucleotide sequence and the oligonucleotide sequence covalently bound to the reactive group. Alternatively, the first probe component and the second probe component (and the third probe component) can comprise two or more pieces that non-covalently associate with one another to produce a functional probe component. The “target binding component” comprises a zipcode oligonucleotide sequence covalently associated with the binding moiety. The “reporter component” comprises an anti-zipcode oligonucleotide sequence that is complementary or substantially complementary to the zipcode oligonucleotide sequence covalently bound to the oligonucleotide sequence of the probe component which itself is covalently bound to the reactive group. The catalytic moiety may be bound to either the target binding component or the reporter component. Preferably, the catalytic moiety is bound to the reporter component. The construction of various zipcode and anti-zipcode oligonucleotides for use in the probe components of this invention is known in the art (see, for example, co-owned International Patent Application Publication Nos. WO 2008/054600 and WO 2006/128138).

Depending upon the biological target and assay format, the first and second (and third, if present) binding moieties may bind to different locations on the biological target (e.g., different nucleotide sequences, different amino acid sequences, different epitopes, etc.). A variety of binding moieties, for example, antibodies, affibodies, adnectins, ligands, receptors, aptamers, nucleic acids, carbohydrates, lipids, small molecules and other binding molecules known in the art can be used in the practice of the invention depending upon the nature of the biological target. Depending upon the target, the binding moieties used in each of the individual first, second (and third) probe components can be the same or different.

In certain embodiments, the reaction product is directly detected because it generates a signal. In this embodiment detection may involve the use of an instrument to detect the level of signal being emitted by the reaction product (e.g, in the case of a fluorescent, chemiluminescent, or colorimetric molecule).

In other embodiments the reaction product itself does not generate a signal and must be detected indirectly through the use of an added agent that both specifically associates with the reaction product and generates a signal. For example, the reaction product may be an antigen that is detected by the addition of a specific antibody that is labeled and the bound antibody is then detected. In certain aspects of this embodiment, the indirect detection of the reaction product results in an amplification of the signal, thus increasing the sensitivity of detection. When an added agent is necessary for detection of the reaction product, that agent interacts preferentially with the reaction products versus the reactive groups. In one aspect of this embodiment, the reaction product is detected using a labeled antibody that either binds directly to the reaction product or binds to a second antibody that binds to the reaction product (e.g., in an ELISA assay).

Other examples of reaction products include a product that: catalyzes a reaction that creates an optical label; inhibits a reaction that creates an optical label; is a fluorescence quencher; is a fluorescent energy transfer molecule; creates a Raman label; creates an electrochemiluminescent label (i.e. ruthenium bipyridyl); produces an electron spin label molecule; is a ligand; is an enzyme activator; or is an enzyme substrate.

The choice of reactive groups for use in the present invention is based upon the following criteria: a) the reactive groups should not react with one another in the absence of the catalyst; b) the reactive groups should produce a product when they react in the presence of the catalyst, where the product can be detected (either directly or indirectly); and c) neither individual reactive group should be detectable by the method used to detect the detectable product.

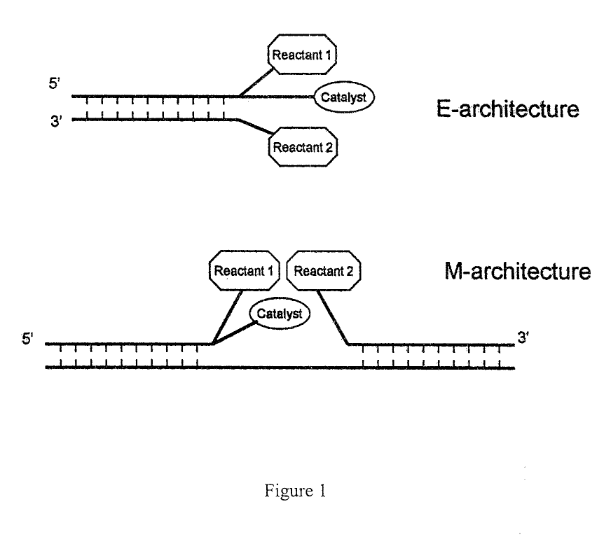

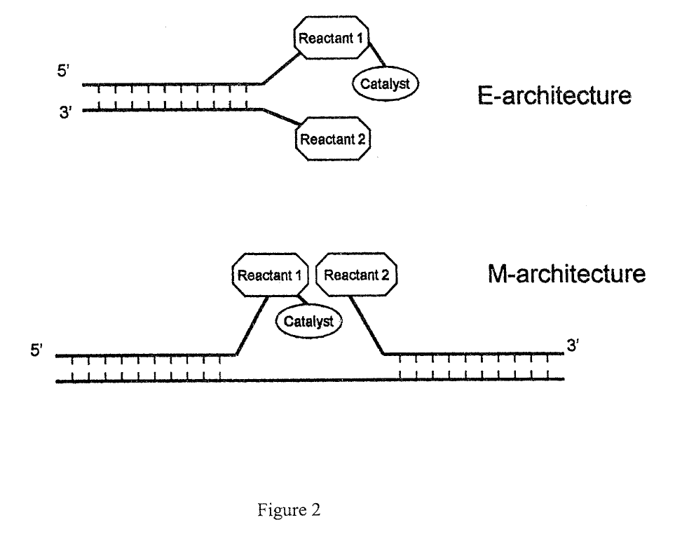

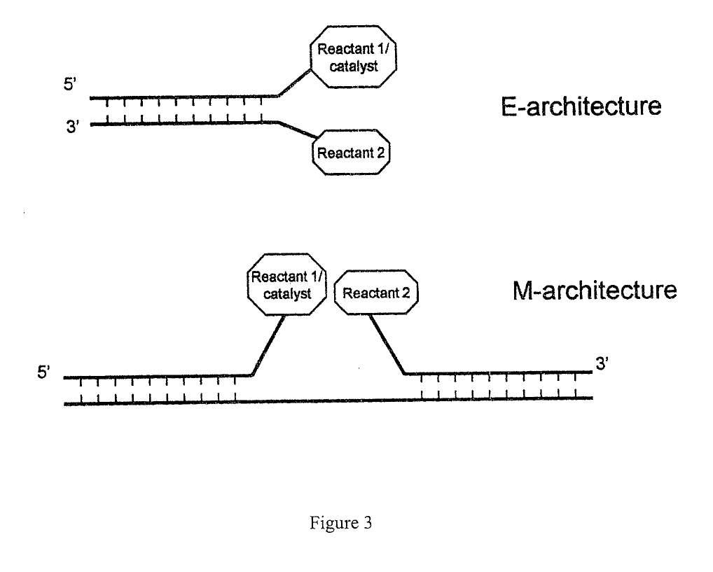

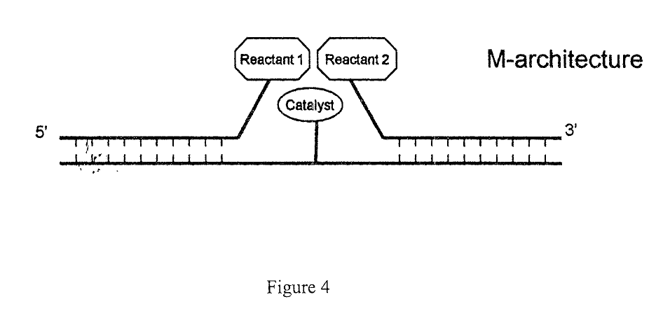

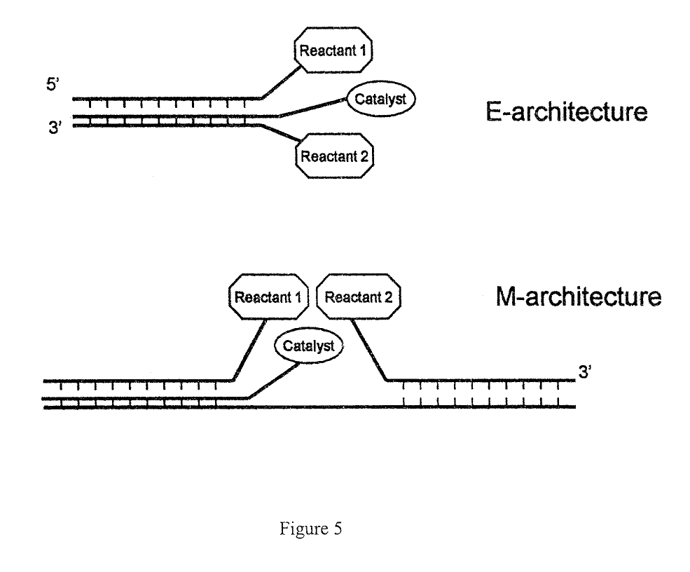

There are several methods that may be employed to conjugate a catalyst into a DNA strand. For example, in Design A (FIG. 1), a catalyst and one of the reactants are linked to a bifurcated DNA while the other reactant is linked to a complementary DNA (end-of-helix, or E-architecture) or a DNA that is complementary to a common template that hydrogen-bond (H-bond) to both DNA reactants (middle-of-helix, or M-architecture). Phosphoramidite containing two orthogonal functional groups can be incorporated into the 5′ end of the DNA for linking the catalyst and the reactant. Extra linkers can be added between the DNA and the catalyst. In Design B (FIG. 2), catalyst and reactant are linked sequentially to a DNA. The sequence of these two molecules relating to the DNA can be switched. Depending on which molecule is linked to the DNA first, the molecule should have two orthogonal functional groups. In Design C (FIG. 3, the catalyst and reactant can be one molecule, which means one part of the molecule can be used as a catalyst and while another part as a reactant. In this case, a molecule containing the functionalities of both a catalyst and a reactant will be synthesized first before linking it to the DNA. In Design D (FIG. 4), catalyst can be linked directly to a DNA template in a M-architecture. A side chain of the nucleobase can be modified to contain a functional group for linking the catalyst. Some of those nucleobase phosphoramidites can be purchased commercially; e.g. amino-modifier C6 dT, carboxy-dT (Glen Research). In Design E (FIG. 5), catalyst is linked to a third DNA strand that can form a triplex with reactant/template strand through Hoogsteen or reversed Hoogsteen hydrogen bonds. In this case, a long stretch of homopurine or homopyridine DNA (over 10 bases) is preferred.

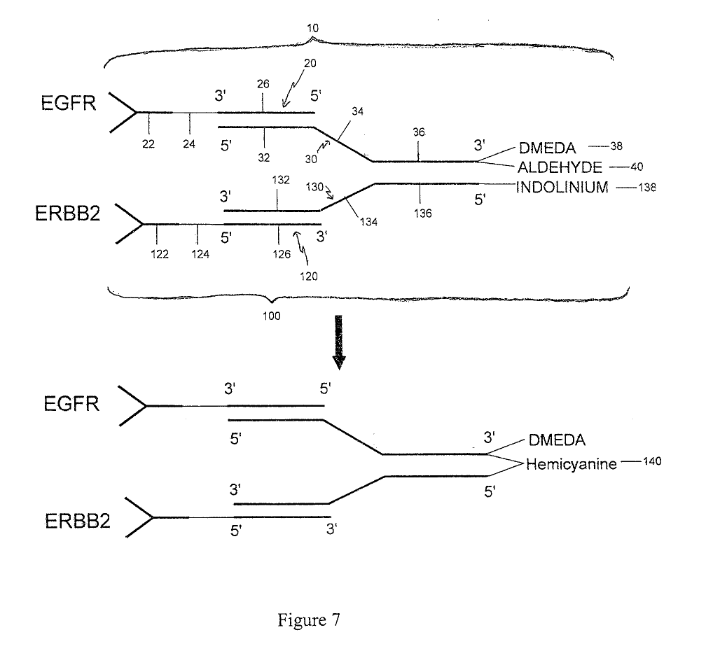

In one embodiment, the methods described herein can be used to detect the formation of a EGFR/ErbB2 heterodimer in a sample of interest. An exemplary system is described in FIG. 7, which shows two probe components, a first probe component denoted 10 that binds to EGFR and a second probe component denoted 100 that binds to ErbB2. Both probe components are two piece ligand reporter assemblies as described, for example, in U.S. patent application Ser. No. 12/176,798, filed Jul. 21, 2008, the contents of which are incorporated by reference herein.

EGFR probe component 10 comprises a target binding component 20 and reporter component 30. Target binding component 20 comprises a first binding moiety 22, for example, an antibody, that specifically binds EGFR linked (either by a covalent bond or through a linker 24) to zipcode 26. Reporter component 30 comprises antizipcode 32 (which is an oligonucleotide sequence complementary to zip code 26) linked (either by a covalent bond or through a linker 34) to a first oligonucleotide 36, which is linked directly or through a linker to (i) a catalyst 38 (for example, a DMEDA catalyst as shown) and (ii) an aldehyde containing reactive group 40. In probe component 10, the target binding component 20 is non-covalently associated with reporter component 30 via hybridization of zipcode 26 and antizipcode 32 to produce a functional probe component.

ErbB2 probe component 100 comprises a target binding component 120 and reporter component 130. Target binding component 120 comprises a first binding moiety 122, for example, an antibody, that specifically binds ErbB2 linked (either by a covalent bond or through a linker 124) to zipcode 126. Reporter component 130 comprises antizipcode 132 (which is an oligonucleotide sequence complementary to zipcode 126) linked (either by a covalent bond or through a linker 134) to a second oligonucleotide 136, which is linked directly or through a linker to an indolinium containing reactive group 138. In probe component 100, the target binding component 120 is non-covalently associated with reporter component 130 by hybridization of zipcode 126 and antizipcode 132 to produce a functional probe component.

If ErbB2 and EGFR have formed a heterodimer, probe components 10 and 100 are brought into proximity with one another. Thereafter, the first oligonucleotide 36 of first probe component 10 hybridizes with second oligonucleotide 136 of second probe component 100 to bring the first reactive group 40 into reactive proximity with the second reactive group 138, which react with one another to produce reaction product 140, which as shown is a hemicyanine. As shown in FIG. 7, catalyst 38 is covalently attached to the first oligonucleotide 36 and catalyzes the production of product 140 from reactive groups 40 and 138. It is understood, however, that a catalyst can be included in probe component 100 rather than in probe component 10 as shown. Furthermore, it is understood that the catalyst can be associated with components other than the first oligonucleotide 36 of probe component 10 or the second oligonucleotide 136 of probe component 100. The presence of reaction product 140 (a fluorophore) is indicative of the presence and/or amount of a EGFR-ErbB2 heterodimer in the sample.

The following examples contain important additional information, exemplification and guidance that can be adapted to the practice of this invention in its various embodiments and equivalents thereof. Practice of the invention will be more fully understood from these following examples, which are presented herein for illustrative purpose only, and should not be construed as limiting in anyway.

### DEFINITIONS

The term, “DNA-programmed chemistry” or “DPC”, as used herein, refers to nucleic acid-templated chemistry, for example, nucleic acid sequence specific control of chemical reactants to yield specific products accomplished by (1) providing one or more templates, which have associated reactive group(s); (2) contacting one or more transfer groups (reagents) having an anti-codon (e.g., complementary sequence with one or more templates) and reactive group(s) under conditions to allow for hybridization to the templates and (3) reaction of the reactive groups to yield products. For example, in a one-step nucleic acid-templated reaction, hybridization of a “template” and a “complementary” oligonucleotide bring together reactive groups followed by a chemical reaction that results in the desired product. Structures of the reactants and products need not be related to those of the nucleic acids comprising the template and transfer group oligonucleotides. See, e.g., U.S. Pat. Nos. 7,070,928 B1 and 7,223,545 and European Patent No. 1,423,400 B1 by Liu et al.; U.S. Patent Publication No. 2004/0180412 (U.S. Ser. No. 10/643,752; Aug. 19, 2003) by Liu et al., by Liu et al.; Gartner, et al., 2004, Science, vol. 305, pp. 1601-1605; Doyon, et al., 2003, JACS, vol. 125, pp. 12372-12373, all of which are expressly incorporated herein by reference in their entireties. See, also, “Turn Over Probes and Use Thereof” by Coull et al., International Patent Application Publication No. WO07/008,276A2, filed on May 3, 2006; “Biodetection by Nucleic Acid-Templated Chemistry” by Coull et al., PCT/US06/20834, WO06/128138A2, filed May 26, 2006.

The terms, “nucleic acid”, “oligonucleotide” (sometimes simply referred to as “DNA” or “oligo”) or “polynucleotide,” as used herein, each refer to a polymer of nucleotides. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA).

The term “detectable reagent” as used herein refers to a reagent that produces a signal that can be observed or quantified by visual observation or through the use of a machine that detects the signal (e.g., a spectrophotometer, a fluorometer, a scintillation counter, a microscope, etc.)

The term “small molecule,” as used herein, refers to an organic compound either synthesized in the laboratory or found in nature having a molecular weight less than 5,000 grams per mole, optionally less than 2,000 grams per mole, and optionally less than 1,000 grams per mole.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

### EXAMPLES

Examples 1-3 below illustrate Designs A, B, and C, and it is contemplated that Designs D and E can be carried out by incorporating the catalysts in a fashion similar to that used to incorporate the reactants.

### Example 1

### Preparation of an Aldehyde and Diamine Linked DNA for Hemicyanine DPC (Design A)

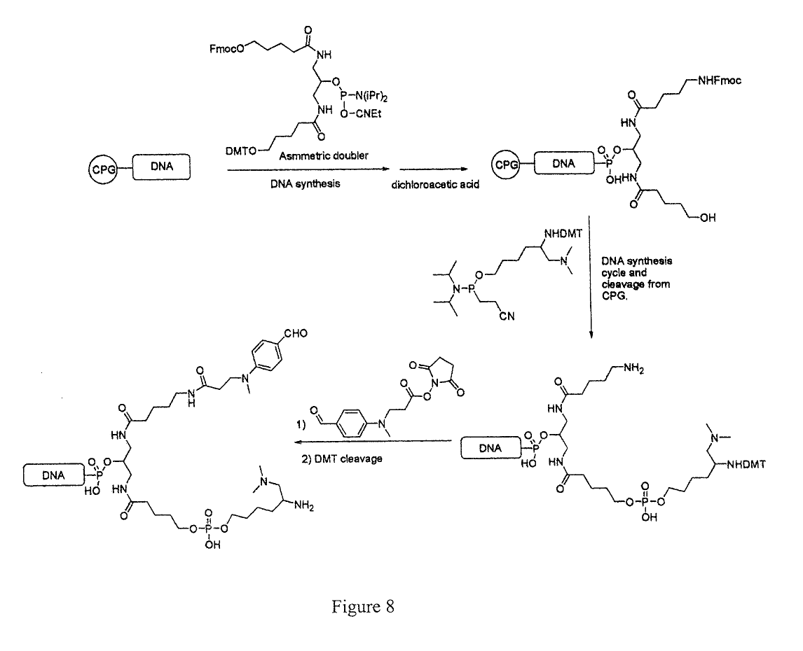

A synthetic sequence for preparing a compound of Design A (i.e., a reactant and catalyst linked bifurcated DNA) is provided in FIG. 8. The synthetic route begins by synthesizing the DNA on a controlled pore glass (CPG) following standard phosphoramidite chemistry. Then, an asymmetric doubler phosphoramidite (based on a dendrimer structure) is introduced. This doubler contains a hydroxyl group protected by an acid-sensitive dimethoxytrityl (DMT) group and an amino group protected by a base-sensitive fluorenylmethoxycarbonyl (Fmoc) group. Upon removing the DMT group using mild acidic conditions (acetic acid), the doubler reacts with the catalyst phosphoramidite. Base-induced cleavage of the DNA from CPG also cleaves the Fmoc group, and the resulting amino group then reacts with aldehyde N-hydroxylsuccimide (NHS) ester to form the desired compound for hemicyanine DPC.

### Example 2

### Preparation of an Aldehyde and Diamine Linked DNA for Hemicyanine DPC (Design B)

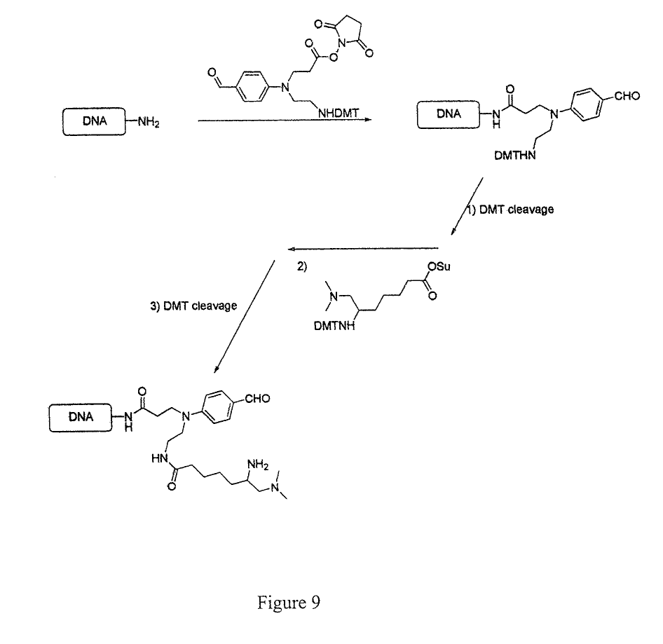

A synthetic sequence for preparing a compound of Design B (i.e., where a reactant and a catalyst are linked to a DNA sequentially) is provided in FIG. 9. The sequence begins by coupling DNA to a phenyl aldehyde compound that includes an NHS ester and DMT-protected amino functional groups. This reaction is typically carried out in solution. After DMT cleavage, the DNA reacts with a diamine NHS ester to form the desired compound for hemicyanine DPC.

### Example 3

### Preparation of an Aldehyde/Diamine Linked DNA for Hemicyanine DPC (Design C)

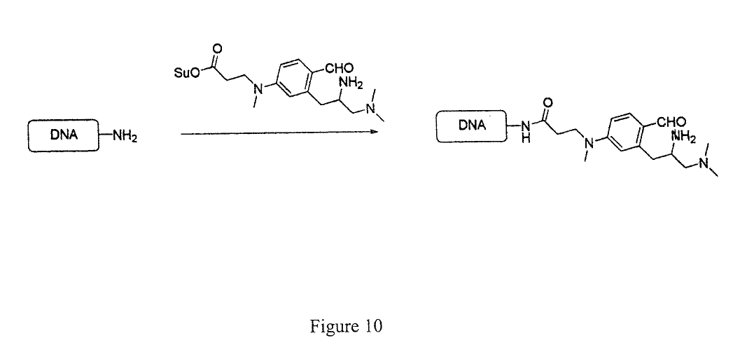

A synthetic sequence for preparing a compound of Design C (i.e., a DNA strand bearing a chemical fragment comprising both a catalyst and a reactant) is provided in FIG. 10. The sequence involves coupling an activated ester to an amino group of the DNA using amide bond-forming conditions.

### Example 4

### Synthesis of a DNA Conjugated Quaternary Salt Bearing Active Hydrogen Component (Indolinium-DNA)

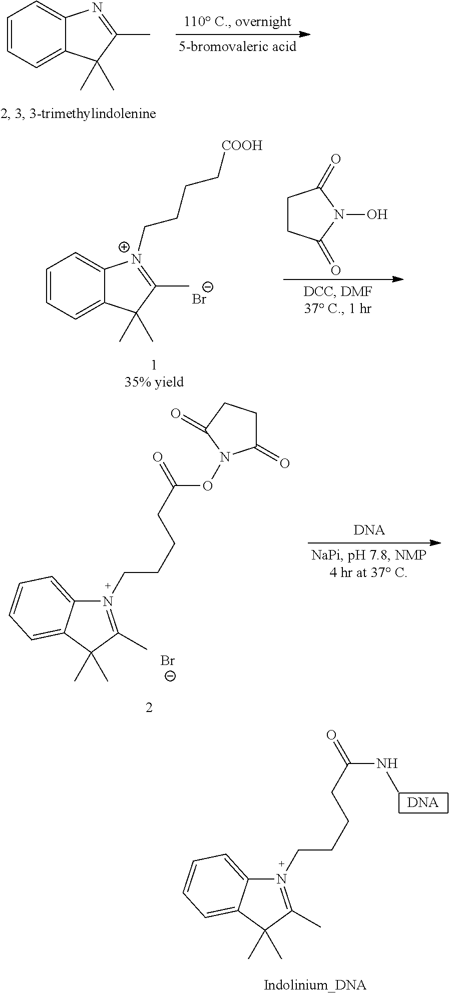

General Experimental Procedures: 2,3,3-trimethylindolenine is commercially available. The acid functionality is introduced to the indoline ring through N-quaternization.

Synthesis of Compound 1: To 5-bromovaleric acid (2.435 g, 13.45 mmole) was added 2,3,3-trimethylindolenine (2.141 g, 13.45 mmole). The reaction mixture was heated with rigorous stirring at 110° C. overnight. The dark red sticky oil obtained was transferred to a Gregar extractor and extracted with EtOAc overnight. A light red solid was obtained. The solid was redissolved in 30 mL of MeOH. MeOH was removed under reduced pressure and the remaining residue was treated with 10 mL of EtOAc. A brownish solid was precipitated out and filtrated. The solid was washed with 2×50 mL of acetone and 2×100 mL of EtOAc. A total 1.590 g of light brownish solid was obtained (35% yield). ¹H NMR (DMSO) δₚₚₘ: 7.98 (m, 1H), 7.84 (m, 1H), 7.61 (m, 2H), 4.49 (t, 2H), 2.84 (s, 3H), 2.30 (t, 2H), 1.84 (m, 2H), 1.63 (m, 2H), 1.53 (s, 6H). MALDI-MS (positive mode): 260.2419.

Synthesis of Compound 2: Compound 1 (0.1 g, 0.294 mmole), N-hydroxy succimide (0.068 g, 0.588 mmole) and N,N′-dicyclohexylcarbodiimide (DCC) (0.085 g, 0.411 mmole) were dissolved in 1.5 mL of dimethyl formamide (DMF). The reaction mixture was stirred at 37° C. for 1 hr. The precipitated dicyclohexylurea (DCU) was removed by filtration, and the filtrate was treated with 15 mL of ether. A light orange solid was washed three times with 10 mL of ether and dried under vacuum for several hours. The solid obtained was used directly for the next reaction. MALDI-MS (positive mode): 357.1590.

Labeling DNA with Indolinium Compound: To a 1.5 mL of centrifugation vial containing 20 nmole of DNA was added 41.6 μL of 0.1 M sodium phosphate buffer (NaPi), pH 7.8, 41.6 μL of compound 2 in N-methyl 2-pyrrolidone (NMP) (96 mM) and 41.6 μL of NMP. The vial was placed in a shaker and shaken for 2 hr at 25° C. The reaction mixture was desalted by gel filtration using Sephadex G-25 and then purified by reversed-phase C8 column. Indolinium_Antizip5: 22% yield. LC-MS (negative mode): Calcd for C₃₀₉H₄₀₂N₁₀₈O₁₇₉P₂₉ (monoisotopic): 1877.1797 [M-6H]⁻⁵; Found: 1877.3717 [M-6H]⁵⁻.

### Example 5

### DNA-Conjugated Aldehyde Containing a Diamine Catalyst

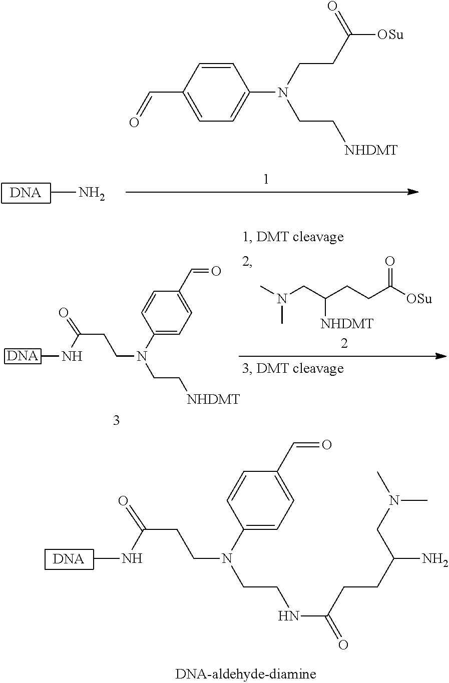

Synthesis of Compound 3: To a 1.5 mL of centrifugation vial containing 20 nmole of DNA was added 41.6 μL of 0.1 M sodium phosphate buffer (NaPi), pH 7.8, 41.6 μL of aldehyde NHS-ester (compound 1) in N-methyl 2-pyrrolidone (NMP) (96 mM) and 41.6 μL of NMP. The vial was placed in a shaker and shaken for 2 hours at 25° C. The reaction mixture was desalted by gel filtration using Sephadex G-25 and then purified by reversed-phase C8 column. The DNA conjugate (compound 3) is dried by lyophilization.

Synthesis of DNA-aldehyde-diamine: The dried DNA is treated with 1 mL of 4:1 acetic acid/water at 4° C. for 1 hour. The solvent is removed by speed vacuum. The DNA is then dissolved in 41.6 μL of 0.1 M sodium phosphate buffer (NaPi), pH 7.8, 41.6 μL of diamine NHS-ester (compound 2) in N-methyl 2-pyrrolidone (NMP) (96 mM) and 41.64 of NMP. The vial was placed in a shaker and shaken for 2 hours at 25° C. The reaction mixture was desalted by gel filtration using Sephadex G-25 and then purified by reversed-phase C8 column. The DNA-aldehyde-diamine conjugate is dried by lyophilization.

The dried DNA is treated with 1 mL of 4:1 acetic acid/water at 4° C. for 1 hour. The solvent is removed by speed vacuum. The residue is dissolved in 1 mL of 2M TEAA solution and purified by reversed-phase C8 column. The DNA is dried by lyophilization.

### Example 6

### Zip-Coded Architecture for DPC-Based Detection of an ErbB (EGFR-ErbB2) Heterodimer in Cells

Compounds described herein (for example, the Indolinium-DNA conjugate described in Example 4 and the DNA-conjugated aldehyde containing a diamine catalyst described in Example 5) can also be used to detect and quantify receptor dimers using a DMEDA-catalyst to facilitate hemicyanine formation. DPC is used to generate a fluorescent hemicyanine signal that depends upon the presence of receptor dimers and effectively discriminates them from constituent monomers.

The optimal design for the binder and reporter oligonucleotides may be achieved by taking into consideration the size and geometry of the binder and the size and geometry of the binding sites of the target. For example, longer or shorter spacer arms may be used to optimally span the distance between binding sites on the target and avoid steric hindrance due to the binders themselves.

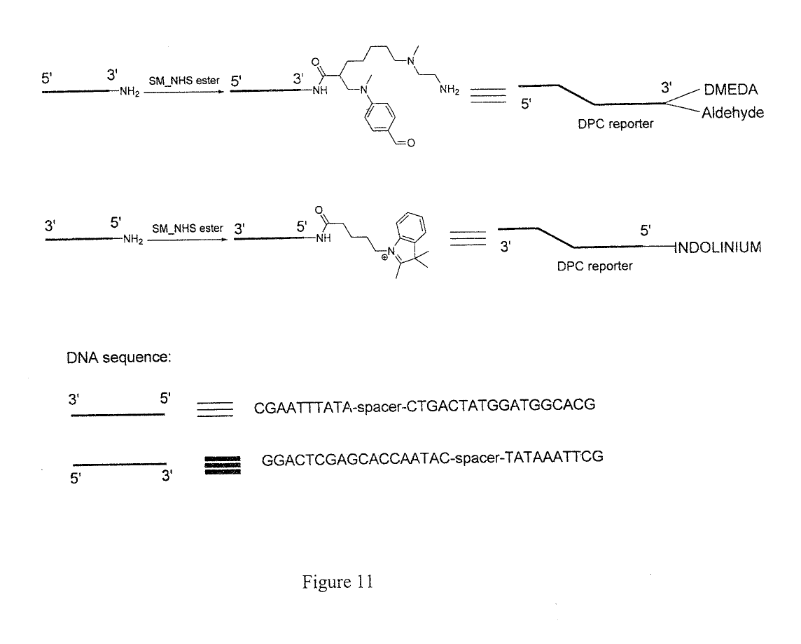

A synthetic sequence for preparing zip-coded oligonucleotides is shown in FIG. 11. A zip-coded oligonucleotide designed to hybridize to the aldehyde-DMEDA reporter molecule is prepared by reacting an activated indolinium compound with a DNA strand having a 5′-amino group. The zip-coded oligonucleotide designed to hybridize to the indolinium reporter molecule is prepared by reacting a DNA strand having a 3′-amino group with a compound containing the catalyst and aldehyde (or protected aldehyde).

Synthesis of the conjugates between oligonucleotides and anti-EGFR or anti-ErbB2 antibody can be performed based on known procedures, such as those described by SoluLink Biosciences (San Diego, Calif.; see www.solulink.com). For example, conjugation of an antibody and oligonucleotides can be carried out by first modifying the primary amino groups of the antibody with succinimidyl 2-hydrazinonicotinate acetone hydrazone to incorporate an acetone hydrazone onto the antibody. Then, the primary amino groups of the oligonucleotides are separately activated with succinimidyl 4-formylbenzoate. The two activated molecules then are mixed in the desired ratio (such as 6:1) and reacted at a mildly acidic pH to form a stable hydrazone linkage. Additional description of synthetic procedures can be found at www.solulink.com.

Two conjugates can be prepared according to this technology: one conjugate containing the zip code to anneal to the DMEDA-aldehyde-containing reporter oligonucleotide, and the other containing the zip code to anneal to the indolinium-containing reporter oligonucleotide.

Antibody-oligonucleotide conjugates prepared according to the above procedures may be further purified by gel chromatography, such as on a 1.6×60 cm column of Superdex S-200 (Amersham Biosciences) in PBS buffer (0.01 M potassium phosphate, pH 7.4-0.138 M sodium chloride). The main antibody peak, eluting at about 0.6 times the column volume, is collected and a later eluting peak of contaminating non-conjugated oligonucleotide may be discarded. The fractions containing the antibody conjugate may be concentrated by reversed dialysis with a Pierce (Rockford, Ill.) 30 K molecular weight cut-off Slide-A-Lyzer using Pierce Concentrating Solution. Protein content can then be determined using a Bio-Rad Micro BCA Reagent Kit, and oligonucleotide content determined using SYBR Gold DNA binding dye (Molecular Probes (Eugene, Oreg.). Purification of the above conjugates according to this procedure provides a conjugate having an average of approximately 3 oligonucleotides per protein molecule.

Assembly of Antibody-Oligo Conjugates with Reporter Oligonucleotides.

The two antibody-oligo conjugates with their reporter (see FIG. 7 and the discussion relating thereto) are first assembled separately in a volume of 10 μL. Each assembly contained 0.5 μM (5 picomoles) of antibody-oligonucleotide conjugate and 0.15 μM of (15 pmoles) of complementary reporter oligonucleotide in 0.05 M Tris/HCl pH 8-0.01 M magnesium chloride. Each is incubated for at least 15 minutes at 4° C. before use in the detection reaction mixture.

### Detection of EGFR Dimers on A431 Cells in Microtiter Plates

EGF-activated A431 cells are washed by centrifugation three times in phosphate buffered saline (“PBS”; Sigma Chemical Company). 50,000 cells are introduced into each well of a hi-bind plate in PBS and allowed to settle overnight at 4° C. The immobilized cells are washed three times with PBS. Wells are blocked with Blocking Solution (PBS-T+1 mg/mL bovine serum albumin+0.1 mg/ml rabbit IgG) for 1 hr at room temperature, then washed three times with PBS plus Tween-20 (“PBS-T”; Sigma Chemical Company), once with water and dried at room temperature.

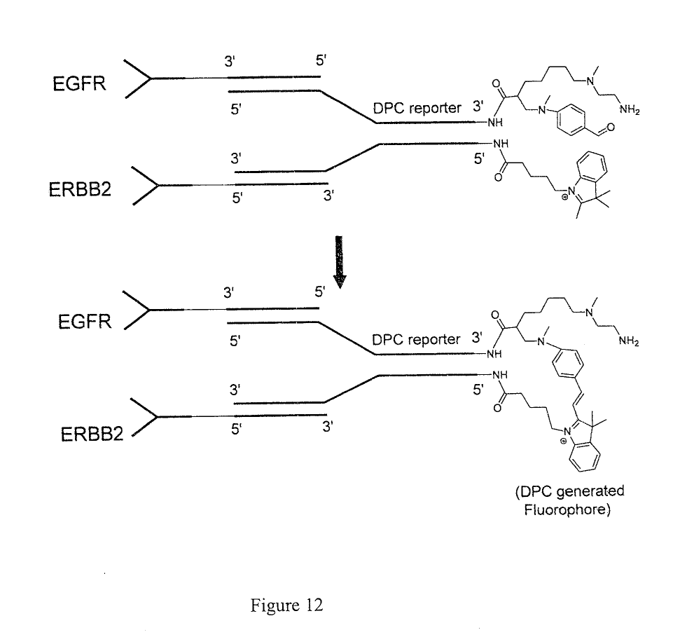

Wells then are incubated with equal amounts of anti-EGFR (Labvision) conjugated to the amino terminus Zip2 (anti-EGFR-Zip2; 0 to 15 pMoles) and anti-ErbB2 (Labvision) conjugated to amino terminus of Zip5 (anti-ErbB2-Zip5; 0 to 15 pMoles) for one hour and washed 3 times with PBS-T (FIG. 12). Samples are incubated in the wells of a 96-well microplate in a fluorimeter at 25° C. for up to 4 hours. Fluorescence can be monitored at various times with excitation at 535 nm and emission at 580 nm.

Alternatively, fluorescence detection of ErbB dimers on A431 cells may be performed by FACS analysis. Cells are treated with EGF as described above, fixed in 3% formaldehyde for 30 min at 4° C., blocked, and incubated with DPC detection reagents as described. In this approach, the drying step would not be necessary.

Controls may be used, such as: a) samples incubated without anti-EGFR conjugates; b) samples incubated without Aldehyde-DMEDA or indolinium conjugates; and c) samples incubated without both anti-EGFR/ErbB2 conjugates Aldehyde or indolinium conjugates.

### INCORPORATION BY REFERENCE

The entire disclosure of each of the publications and patent documents referred to herein is incorporated by reference in its entirety for all purposes to the same extent as if each individual publication or patent document were so individually denoted.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method for detecting a biological target in a sample, the method comprising:
(a) providing a first probe component comprising (i) a first binding moiety having binding affinity to the biological target, (ii) a first oligonucleotide sequence associated with the first binding moiety, (iii) a first reactive group associated with the first oligonucleotide sequence, and (iv) an optional catalytic moiety associated with the first probe component;
(b) providing a second probe component comprising (i) a second binding moiety having binding affinity to the biological target, (ii) a second oligonucleotide sequence associated with the second binding moiety, (iii) a second reactive group associated with the second oligonucleotide sequence, and (iv) an optional catalytic moiety associated with the second probe component, wherein the second oligonucleotide is capable of hybridizing to the first oligonucleotide sequence and the second reactive group is capable of reacting with the first reactive group, and wherein at least one catalytic moiety is associated with the first probe component or the second probe component and the catalytic moiety is capable of catalyzing the reaction between the first reactive group and the second reactive group;
(c) combining the first and second probe components with a sample to be tested for the presence of the biological target under conditions that permit (i) the first binding moiety and the second binding moiety to bind to the biological target, if present in the sample, and (ii) the second oligonucleotide to hybridize to the first oligonucleotide thereby to bring the first reactive group into reactive proximity with the second reactive group; and
(d) detecting a reaction between the first and the second reactive groups thereby to determine the presence of the biological target in the sample.

2. The method of claim 1, wherein the biological target is a protein and wherein each of the first binding moiety and the second binding moiety is an antibody.

3. The method of claim 1, wherein the first binding moiety and second binding moiety bind different sites on the biological target.

4. The method of claim 3, wherein the biological target is selected from an ErbB protein family homodimer, an ErbB protein family heterodimer, a VEGF receptor homodimer, a VEGF receptor heterodimer, a VEGF dimmer, a PDGF dimmer, a tyrosine kinase receptor complex, a TNF/TNFR complex, a cadherin complex, a catenin complex, an IGFR complex, an insulin receptor complex, an EPO/EPO receptor complex, a NF-kB/IkB complex, a T-cell antigen complex, an integrin protein complex, a FKBP protein complex, a p53 protein complex, a Bcl family protein complex, a Myc/Max complex, a cyclin protein complex, an intracellular protein kinase complex, a caspase protein complex, an autoantibody-antigen complex, an amyloid protein complex, a Bcr-Abl fusion protein and a NPM-ALK fusion protein or another ALK-containing fusion protein.

5. The method of claim 4, wherein the biological target is selected from an ErbB protein family homodimer and an ErbB protein family heterodimer.

6. The method of claim 1, wherein the biological target is a nucleic acid and wherein each of the first binding moiety and the second binding moiety is a nucleic acid that is complementary to a portion of the biological target.

7. The method of claim 1, wherein the catalytic moiety is a diamine of the formula (II): 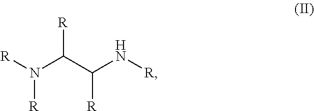
wherein each R is independently selected from hydrogen or C₁-C₆ straight or branched alkyl.

8. The method of claim 7, wherein the catalytic moiety is selected from N¹,N¹-dimethylethane-1,2-diamine, N¹,N¹-dimethylpropane-1,3-diamine, N²,N²-dimethylpropane-1,2-diamine, ethylenediamine, N¹,N¹-diethylethylene-1,2-diamine (“DMEDA”), propane-1,2-diamine, and 1-(2-aminoethyl)-piperidine.

9. The method of claim 8, wherein the catalytic moiety is DMEDA.

10. The method of claim 1, wherein the first oligonucleotide is covalently associated with the first binding moiety and the second oligonucleotide is covalently associated with the second binding moiety.

11. The method of claim 1, wherein the first oligonucleotide is non-covalently associated with the first binding moiety and the second oligonucleotide is non-covalently associated with the second binding moiety.

12. The method of claim 1, wherein the catalytic moiety is covalently associated with the first oligonucleotide or the second oligonucleotide.

13. The method of claim 1, wherein the catalytic moiety is covalently associated with the first reactive group or the second reactive group.

14. A method of performing a chemical reaction using a nucleic acid template to produce a reaction product, the method comprising the steps of:
(a) providing (i) a template comprising a first reactive group covalently associated to a first oligonucleotide defining a first codon sequence and an optional catalytic moiety covalently associated with the first oligonucleotide or the first reactive group, and (ii) a transfer unit comprising a second reactive group covalently associated with a second oligonucleotide defining a first anti-codon sequence complementary to the first codon sequence of the template and an optional catalytic moiety covalently associated with the second oligonucleotide or the second reactive group, wherein at least one catalytic moiety is covalently associated with one of the first oligonucleotide, the second oligonucleotide, the first reactive group, or the second reactive group; and
(b) combining the template and the transfer unit under conditions so that the first codon sequence and the first anti-codon sequences anneal to one another thereby to bring the first reactive group into reactive proximity with the second reactive group whereupon the first reactive group reacts with the second reactive group to produce a reaction product, wherein the reaction between the first reactive group and the second reactive group is catalyzed by the catalytic moiety.

15. A method for performing a chemical reaction using a nucleic acid template to produce a reaction product, the method comprising the steps of:
(a) providing (i) a first transfer unit comprising a first oligonucleotide sequence and a first reactive group covalently associated with the first oligonucleotide sequence and an optional catalytic moiety covalently associated with the first oligonucleotide or the first reactive group, (ii) a second transfer unit comprising a second, different oligonucleotide sequence and a second reactive group covalently associated with the second oligonucleotide sequence, and an optional catalytic moiety covalently associated with the second oligonucleotide or the second reactive group, and (iii) a template comprising a template oligonucleotide sequence and an optional catalytic moiety covalently associated with the template oligonucleotide, wherein the first oligonucleotide sequence and the second oligonucleotide sequence are complementary to two separate regions of the template oligonucleotide, and wherein at least one catalytic moiety is covalently associated with one of the first oligonucleotide, the second oligonucleotide, the first reactive group, the second reactive group, or the template oligonucleotide;
(b) combining the first transfer unit, the second transfer unit and the template under conditions so that the first oligonucleotide and the second oligonucleotide hybridize to their respective complementary regions of the template oligonucleotide thereby to bring first reactive group into reactive proximity with the second reactive group whereupon the first reactive group reacts with the second reactive group thereby to produce a reaction product, wherein the reaction between the first reactive group and the second reactive group is catalyzed by the catalytic moiety.

16. A method of performing a chemical reaction using a nucleic acid template to produce a reaction product, the method comprising the steps of:
(a) providing (i) a template comprising a first reactive group covalently associated with a first oligonucleotide defining a first codon sequence, (ii) a transfer unit comprising a second reactive group associated with a second oligonucleotide defining a first anti-codon sequence complementary to the first codon sequence of the template, and (iii) a third unit comprising a catalytic moiety covalently attached to a third oligonucleotide defining a third sequence, wherein the third oligonucleotide is capable of forming a triplex with the first and the second oligonucleotides through Hoogsteen or reversed Hoogsteen hydrogen bonds; and
(b) combining the template, the transfer unit, and the third unit under conditions to produce a triplex comprising the first, second and third oligonucleotides where upon the catalytic moiety, the first reactive group and the second reactive group are brought into reactive proximity so that the first and second reactive groups react with one another to produce the reaction product and the reaction between the first and the second reactive groups is catalyzed by the catalytic moiety.

17. A method for performing a chemical reaction using a nucleic acid template to produce a reaction product, the method comprising the steps of:
(a) providing (i) a first transfer unit comprising a first oligonucleotide sequence and a first reactive group covalently associated to the first oligonucleotide sequence, (ii) a second transfer unit comprising a second oligonucleotide sequence and a second reactive group covalently associated to the second oligonucleotide sequence, (iii) a template comprising a template oligonucleotide sequence, wherein the first oligonucleotide sequence and the second oligonucleotide sequence are complementary to two separate regions of the template oligonucleotide, and (iv) a third unit comprising a catalytic moiety covalently associated with a third oligonucleotide defining a third sequence, wherein the third oligonucleotide is capable of forming a triplex with the first oligonucleotide and the template oligonucleotide through Hoogsteen or reversed Hoogsteen hydrogen bonds; and
(b) combining the first transfer unit, the second transfer unit, the template and the third unit under conditions so that (i) the first oligonucleotide and the second oligonucleotide hybridize to their respective complementary regions of the template oligonucleotide thereby to bring the first reactive group into reactive proximity with the second reactive group, and (ii) the first oligonucleotide, the template oligonucleotide and the third oligonucleotide form a triplex to bring the catalytic moiety into proximity with the first and second reactive groups so that the first and second reactive groups react to produce a reaction product, and the reaction between the first and the second reactive groups is catalyzed by the catalytic moiety.

18. A method for detecting a biological target, the method comprising:
(a) providing a first probe component comprising (i) a first binding moiety having binding affinity to the biological target, (ii) a first oligonucleotide sequence, and (iii) a first reactive group covalently associated with the first oligonucleotide sequence;
(b) providing a second probe component comprising (i) a second binding moiety having binding affinity to the biological target, (ii) a second oligonucleotide sequence, and (iii) a second reactive group associated with the second oligonucleotide sequence, wherein the second oligonucleotide is capable of hybridizing to the first oligonucleotide sequence and the second reactive group is capable of reacting with the first reactive group when the second reactive group is brought into reactive proximity with the first reactive group;
(c) providing a third probe component comprising (i) a third binding moiety having binding affinity to the biological target, (ii) a third oligonucleotide sequence, and (iii) a third reactive group reactive with the first and/or the second reactive groups, or a catalytic moiety, wherein the catalytic moiety is capable of enhancing the reaction rate between the first and second reactive groups;
(d) combining the first, the second and the third probe components with a sample to be tested for the presence of the biological target under conditions where the first, the second and the third binding moieties bind to the biological target, if present in the sample;
(e) allowing the second oligonucleotide to hybridize to the first oligonucleotide to bring first reactive group into reactive proximity with the second reactive group; and
(f) forming a triplex between the first, the second and the third oligonucleotides thereby to bring the first and second reactive groups into proximity with the third reactive group or the catalytic moiety; and
(g) detecting a reaction among the reactive groups thereby to determine the presence of the biological target.

19. An improved method of detecting a biological target in a sample, wherein the improvement comprises producing a reaction product by the method of claim 14, wherein the formation of the reaction product is indicative of the presence of the biological target in the sample.

20. The method of claim 19, wherein the biological target is selected from an ErbB protein family homodimer and an ErbB protein family heterodimer.


SEQUENCE LISTING (4 sequences)

SEQ ID NO: 1 - DNA, 10 bp, Artificial Sequence
  Description of Artificial Sequence Synthetic oligonucleotide
cgaatttata

SEQ ID NO: 2 - DNA, 18 bp, Artificial Sequence
  Description of Artificial Sequence Synthetic oligonucleotide
ctgactatggatggcacg

SEQ ID NO: 3 - DNA, 18 bp, Artificial Sequence
  Description of Artificial Sequence Synthetic oligonucleotide
ggactcgagcaccaatac

SEQ ID NO: 4 - DNA, 10 bp, Artificial Sequence
  Description of Artificial Sequence Synthetic oligonucleotide
tataaattcg